(19) United States
(12) Patent Application Publication
(10) Pub. No.: US 2020/0319160 A1
(43) Pub. Date: Oct. 8, 2020

(54) DETERMINING A GAS CONCENTRATION

(71) Applicant: Robert Bosch GmbH, Stuttgart (DE)

(72) Inventors: Richard Fix, Weil Im Schoenbuch (DE); Christoph von Jutrzenka Trzebiatowski, Dresden (DE); Philipp Nolte, Gerlingen (DE)

(73) Assignee: Robert Bosch GmbH, Stuttgart (DE)

(21) Appl. No.: 16/300,702

(22) PCT Filed: May 12, 2017
(86) PCT No.: PCT/EP2017/061456

§ 371 Date: Nov. 12, 2018

(30) Foreign Application Priority Data
Jun. 3, 2016 (DE) 102016209802.0

(51) Int. Cl. G01N 33/497; G01N 33/00; A61B 10/00

(52) U.S. Cl. CPC G01N 33/4972; G01N 33/0031; G01N 33/007; A61B 2010/0087; A61B 2010/0009; A61B 2010/0083; A61B 10/00

(57) ABSTRACT

A device for determining the concentration of a predefined gas in a medium includes a first sensor for determining the gas concentration in a flow of the medium; a second sensor for determining a physical parameter of the medium in the flow of the medium; and a processing device, which is set up to determine the quality of the determination using the first sensor on the basis of a signal from the second sensor.

### BACKGROUND INFORMATION

The present invention relates to the determination of a gas concentration. In particular, the present invention relates to the determination of a gas concentration with the aid of a free-jet measuring device.

A mobile device includes a sensor for determining a gas concentration. For example, the mobile device may be set up to determine the concentration of gaseous alcohol in respiratory air. A more sophisticated analyzing device normally includes a mechanical device by which a predefined oncoming flow of respiratory air to the gas sensor is induced, e.g., a mouthpiece or a pump. A less expensive analyzing device frequently operates according to the free-jet measuring principle in which a predefined oncoming flow of the gas sensor is unable to be forced. To determine the gas concentration, the gas sensor must normally be brought to a specific operating state or to a sequence of predefined operating states during a measuring operation, which requires a predefined measuring period, in order to successfully conclude the measurement. If a predefined oncoming flow of the gas sensor by the medium to be measured fails to take place, then the measurement may be inaccurate or unusable.

### SUMMARY

An object of the present invention is to provide a technique by which a determination of a gas concentration is able to be carried out in a more optimal manner, in particular with the aid of a free-jet gas sensor. Preferred specific embodiments are described herein.

In accordance with an example embodiment of the present invention, a device for determining the concentration of a predefined gas in a medium (gas concentration) includes a first sensor for determining the gas concentration in a flow of the medium; a second sensor for determining a physical parameter of the medium in the flow of the medium; and a processing device, which is set up to determine the quality of the determination by the first sensor on the basis of a signal from the second sensor.

The starting point is a free-jet sensor whose position in the flow of the medium is not forced by additional measures. For this purpose, the sensor is usually fixed in place on the device, and no pump, fan or closed pipe system for inducing induce a volume flow of the medium past the sensor is provided. The medium is usually gaseous, and the predefined gas is preferably homogeneously mixed with the medium. Preferably, the predefined gas includes a volatile organic compound (VOC). This makes it possible to detect solvents such as formaldehyde, which may be contained in varnishes, paint strippers, cleaning substances, furniture, office equipment, adhesives or alcoholic substances. In addition, it is possible to detect compounds that are contained in expelled respiratory air and which may provide an indication of an illness or the metabolism of a person.

The physical parameter may particularly relate to a humidity, a pressure or a temperature of the medium. This particularly makes it possible to monitor the incoming flow behavior of the medium toward the first sensor. For this purpose, the first and the second sensors are preferably disposed in close physical proximity to each other and may particularly be integrated with each other in a shared housing. A suitable sensor is able to be obtained under the designation BME680 in a housing of 3×3×0.95 mm³.

On the one hand, the determination of the measuring quality makes it possible to monitor whether an incoming flow of the first sensor that leads to a usable result has been present during a required measuring period. On the other hand, the determined quality may be used to interpret a determined gas concentration, for instance in that a measuring uncertainty is indicated as well, or in order to already make a statement about the determination quality during the measurement, so that an operator is able to influence the measuring conditions.

The device may particularly be disposed in a mobile device, which is either specifically set up to determine the gas concentration or is installed in another mobile device such as a smartphone or a tablet computer. Especially the second sensor may either already be provided in the mobile device or else may also be enabled for other purposes within the mobile device, thereby resulting in a greater device utilization as a whole.

It is preferred that the processing device is furthermore set up to control the determination of the gas concentration by the first sensor as a function of the determined quality.

As a rule, the first sensor must be operated in a certain manner in order to allow for the determination of the gas concentration. For example, it may be necessary to sequentially heat the first sensor to a series of predefined temperatures. This is also known as a measuring profile, which is normally controlled externally and preferably by the processing device.

The measuring profile may preferably be started only when the determined quality lies above a predefined threshold value. In addition, the measurement may be found successful and be concluded when a threshold value of a minimally required quality is undershot during the measuring period.

It is particularly preferred that a plurality of second sensors is provided, which are set up to determine different physical parameters of the medium in the flow, the processing device being set up to determine the quality of the determination on the basis of the signals from the second sensors.

The more physical parameters of the medium are detected in the flow, the more precise the incoming flow behavior of the medium is able to be reproduced or assessed. Further possible parameters may include a dewpoint or a viscosity, for example.

In one further specific embodiment, a third sensor is provided for determining a physical parameter in the environment of the device. For this purpose, the processing device is set up to determine the quality of the determination additionally also on the basis of the signal from the third sensor.

The third sensor may particularly provide information about the handling of the device. For example, using an acceleration sensor, a yaw-rate sensor or a magnetic sensor, it is possible to determine whether the mobile device is held steady during the measuring period. If the third sensor includes a microphone, then a flow noise of the medium is able to be evaluated. Background noises are alternatively or additionally able to be evaluated in an effort to determine whether an environment was selected for the measurement that may possibly not be quiet enough. If multiple microphones are available, then the direction of an incoming flow is able to be determined using rotational angulation. An optical sensor, e.g., a proximity sensor or a light sensor, may be used to check the handling of the device in greater detail. For example, it is able to be determined whether a distance between a user and the device is being changed during a respiratory air analysis.

It is furthermore preferred that the processing device is set up to output an item of information regarding the determined quality. An output may particularly take place when the determined quality is insufficient to fully carry out a meaningful determination of the gas concentration. Especially when multiple sequential determination attempts exhibit insufficient qualities, a further measurement is able to be carried out using a shortened measuring profile. A resulting reduced determination quality is able to be supplied together with the determined gas concentration.

In addition, the processing device may be set up to supply an item of information regarding an improved handling of the device on the basis of the determined quality and a signal from the second and/or the afore-described third sensor.

This item of information may be output acoustically, optically or also haptically, for example. In one specific embodiment, the item of information relates only to one handling parameter or to a combination of multiple handling parameters. The handling parameters may include a position of the device in space or a position relative to the user, for example.

A method for determining the concentration of a predefined gas in a medium includes steps of determining the gas concentration in a flow of the medium with the aid of a first sensor, of determining a physical parameter of the medium in the flow of the medium with the aid of a second sensor, and of determining, on the basis of a signal from the second sensor, the quality of the determination by the first sensor.

The present method may particularly be carried out with the aid of the afore-described device.

In one specific embodiment, the determination of the gas concentration is found to be successful when a physical parameter of the medium lies within a predefined range during a measuring process with the aid of the first sensor. For this purpose, a lower limit and/or an upper limit may be specified for the physical parameter. The measuring operation requires a predefined measuring period during which one of the limits of the predefined range is able to be adapted.

In one further specific embodiment, a derivation of the physical parameter of the medium as a function of time is evaluated additionally or alternatively. A range may correspondingly be provided for the derivation, and the range limits may be variable via the measuring period.

In a still further specific embodiment, a higher derivation of a physical parameter of the medium as a function of time is evaluated. Here, too, a range may be predefined in which the higher derivation of the parameter must lie for the determination of the gas concentration to be considered successful.

### BRIEF DESCRIPTION OF THE DRAWINGS

The present invention is described in greater detail with reference to the figures.

FIG. 1 shows a device for determining a gas concentration in a medium.

FIG. 2 shows characteristics of physical parameters at the device from FIG. 1.

FIG. 3 shows a measuring profile of a sensor at the device from FIG. 1.

FIG. 4 shows a flow diagram of a method for determining a gas concentration with the aid of the device from FIG. 1.

### DETAILED DESCRIPTION OF EXAMPLE EMBODIMENTS

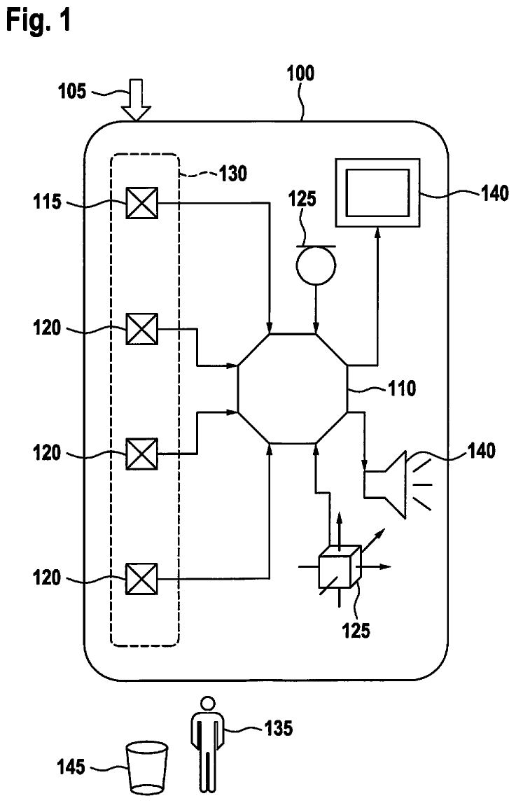

FIG. 1 shows a device 100 for determining a gas concentration in a medium 105. Device 100 may particularly be part of a mobile device such as a smartphone. Device 100 includes a processing device 110, a first sensor 115, which is set up to determine a concentration of a gas in medium 105, one or more second sensor(s) 120, which is/are developed to determine different physical parameters of medium 105, and preferably one or more third sensor(s) 125, which is/are developed to detect a physical parameter in the environment of device 100 or in the environment of first sensor 115 in each case. For example, third sensor 125 may include an acceleration sensor, a yaw-rate sensor, a magnetic-field sensor or a position sensor. In the illustrated preferred specific embodiment, first sensor 115 and up to three second sensors 120 are integrated in a shared sensor system 130. In this particular example, second sensors 120 relate to a temperature, a pressure, and a relative humidity of medium 105.

First sensor 115 is preferably a free-jet sensor, which means that it has no active devices such as a pump or a fan which ensure a flow of medium 105 in a predefined manner. Generally, there is also no passive device provided such as a tube, a hose, or a throttle for shaping the flow of the medium. Medium 105 may therefore approach first sensor 115 from different directions and in different manners, which means that measuring conditions are able to be controlled or determined only with difficulty.

Third sensors 125 are preferably situated directly on device 100 but need not necessarily rest very tightly against sensors 115 and 120. Third sensors 125 may particularly determine the handling of device 100 with regard to a position in space, a vibration, an alignment, or an acceleration. In addition, using third sensors 125, an environment of device 100 is able to be characterized. For example, it may at least approximately be determined whether a person 135 or multiple persons 135 is/are present in the region of device 100, whether device 100 is located in an enclosed space, and what time of day it is.

In one further specific embodiment, a flow behavior of medium 105 is able to be inferred with the aid of one or a plurality of third sensor(s) 125. For example, the flow of medium 105 may be traced with the aid of a microphone 125.

Device 100 may also be equipped with one or more output means 140. Purely by way of example, a screen is shown as an optical output means in the representation of FIG. 1, and a loudspeaker as an acoustic output means. Furthermore, a haptic output means such as a vibration motor, or also some other output means 140 may be provided. Output means 140 may be used to supply the result of the determination of the gas concentration in medium 105. Moreover, using an output means 140, an item of information regarding a determination quality is able to be output, i.e., during or following the determination. In addition, operating instructions for device 100 may be output in order to induce an operator to provide optimum measuring conditions, if possible.

Device 100 is able to be used for a number of different purposes. If first sensor 115 includes a sensor for volatile organic compounds, then it may be used for analyzing the respiratory air of person 135, for instance. In one specific embodiment, an alcohol content in the exhaled air 105 of person 135 is analyzed. It can then be determined whether person 135 is competent to drive. In other specific embodiments, halitosis (bad breath) is able to be determined, or an indicator of a fatty acid catabolism (ketosis), an asthma indicator (FeNO), an indicator of fructose tolerance (H₂), or some other parameter of person 135. In a still further specific embodiment, odors of a food or a drink 145 may be analyzed in order to determine ingredient components. Here, too, the determination of an alcohol content is able to be carried out on the basis of the odors.

When the respiratory air of person 135 is analyzed, attention should usually be paid that the largest possible portion of the expellable lung volume is guided past first sensor 115 in a uniform manner and at a predefined angle. When determining odors of the food or drink 145, the most uniform convection possible between drink 145 and first sensor 115 is normally endeavored.

It is provided to set up processing device 110 for determining a quality of the determination of the gas concentration with the aid of first sensor 115 on the basis of second sensor(s) 120 and possibly third sensor(s) 125. In one specific embodiment, the determination quality is able to be output or used for interpreting the determined gas concentration. In one further specific embodiment, processing device 110 may control the determination of the gas concentration by first sensor 115 on the basis of the determined quality.

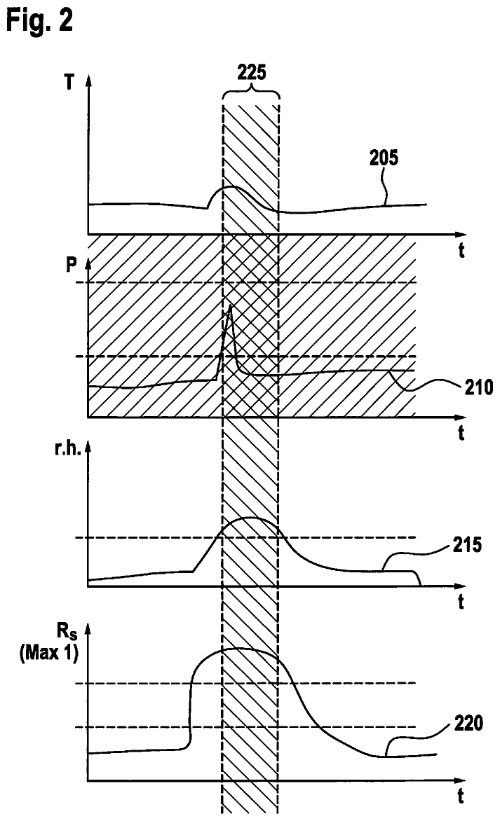

FIG. 2 shows exemplary characteristics of physical parameters on device 100 from FIG. 1. In an exemplary manner, it is assumed that a respiratory air analysis of person 135 with the aid of device 100 forms the basis of the illustrated characteristics.

A first characteristic 205 relates to the temperature of medium 105. Due to the exhalation of person 135, the determined temperature of medium 105 rises. A minimum temperature may be predefined as a starting condition for the measurement of the gas concentration by first sensor 115. In addition, it may be required that the temperature lie within a predefined range during the measurement. A temperature that is too low may lead to a condensation of components of medium 105 and thus to a falsification of the measuring result.

A second characteristic 210 relates to a pressure of medium 105. When person 135 exhales, the pressure will initially exhibit a strong rise, but it drops back to the initial level again after a short period of time. Person 135 may possibly have exhaled only briefly in the direction of allocated second sensor 120 but has done so with sufficient intensity to enrich a volume that surrounds first sensor 115 with respiratory air. This then makes it possible to infer characteristics 215 and 220 that will be examined in the following text.

A third characteristic 215 relates to a relative humidity of medium 105. The relative humidity already begins to rise before the pressure of second characteristic 210 has reached its maximum. Illustrated characteristic 215 points to an early, not very intensive exhalation of person 135 or to a diffusion of the humid respiratory air in the direction of the gas chamber inside which allocated second sensor 120 is located. The illustrated humidity values lie within a predefined expected range. Exhaled air is normally saturated, i.e. it contains a high relative humidity. If the relative humidity exceeds a predefined threshold value, then it may be inferred that medium 105 in the region of second sensor 120 includes exhaled air of person 135.

A fourth characteristic 220 relates to the gas concentration determined with the aid of first sensor 115. Fourth characteristic 220 is similar to third characteristic 215 and may therefore be considered plausible.

Conditions that appear to make a determination of the gas concentration meaningful are present within a time period 225. Accordingly, first sensor 115 may be controlled for the purpose of determining the gas concentration.

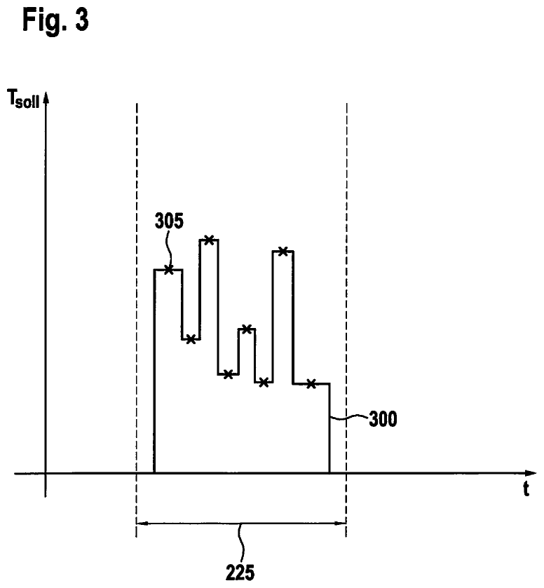

FIG. 3 shows a measuring profile 300 of first sensor 115 on device 100 from FIG. 1. Measuring profile 300 shows an exemplary parameter to which first sensor 115 must be brought in order to carry out a predefined measurement. In the case at hand, this parameter includes a temperature, which may lie in a range of up to multiple 100° C. Within time period 225, which is determined on the basis of second sensors 120 and possibly one or more third sensor(s) 125, first sensor 115 is brought to different measuring points 305 in sequence in order to carry out the required determinations. The determination of a gas concentration is able to be performed at each measuring point 305. The type of the determined gas may be a function of the controlled temperature.

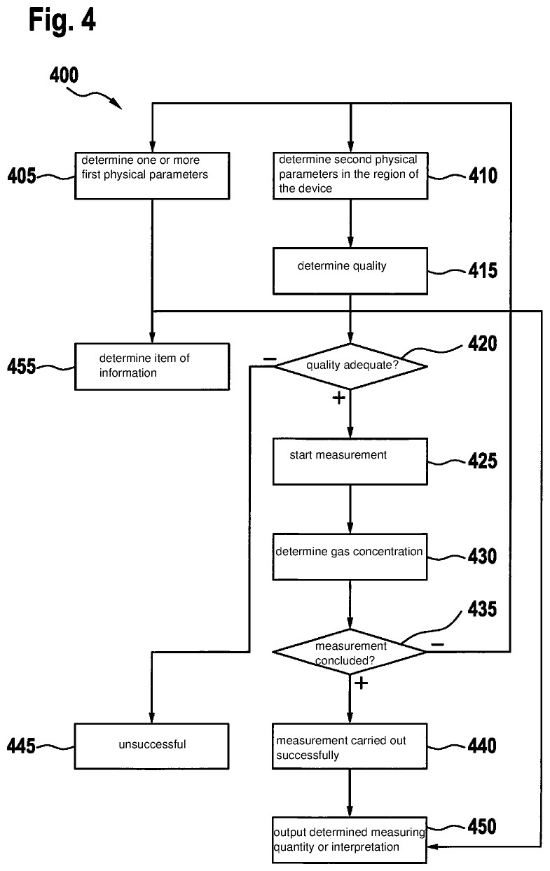

FIG. 4 shows a flow diagram of a method 400 for determining a gas concentration with the aid of device 100 from FIG. 1. Method 400 is particularly developed for running on device 100 or on processing device 110.

In a step 405, one or more first physical parameter(s) of medium 105 is/are determined with the aid of a second sensor 120 or multiple second sensors 120. In parallel therewith, a determination of second physical parameters in the region of device 100 may take place in a step 410 using one or more third sensor(s) 125.

On the basis of the determined parameters, a determination quality is determined in a step 415. The quality indicates whether characteristics 205 through 220 of output signals from sensors 115, 120, 125 are plausible for carrying out a measurement. Plausibility means that a sensor signal, its single time derivation or its multiple time derivations lie(s) within a predefined range. Alternatively or additionally, the signals may be put into context with one another in order to ensure that no contradictions are present.

In a step 420, it is determined that the quality is adequate, so that the measurement is able to be started in a step 425. In a step 430, the gas concentration is determined with the aid of first sensor 115. For this purpose, first sensor 115 may be brought to a predefined measuring point 305 of a measuring profile 300 (see FIG. 3). In a step 435, it is checked whether the measurement has already been concluded. If this is not the case, then method 400 may return to the beginning and be run through again. In the other case, it is determined in a step 440 that the measurement has been carried out successfully, and the determination result may be output in a step 450. In addition, a determined measuring quality is able to be output or an interpretation of the measurement on the basis of the determined quality may take place.

If it is determined during the measurement in step 420 that at least one sensor signal or its derivation lies outside a predefined range or that two or more signals are unable to be plausibilized with one another, then method 400 may transition to a state 445 in which the measurement is assessed as unsuccessful.

From state 445, an analysis of individual signals from sensors 115 through 125 is able to be carried out in order to determine the reasons for the negative validity check. For example, an incorrect position of device 100, blowing at an incorrect angle with regard to device 100 during the breath analysis, or a dirty filter upstream from one of sensors 115 through 125 may be detected. Thereafter, information regarding the unsuccessful measurement or information as to the manner in which the measuring conditions could be improved so that a valid measurement is able to be carried out may be output with the aid of output means 140. A graphical indication, in particular, may be output, also an animated one, in order to illustrate the correct handling of device 100. Furthermore, a prompt for a repeat of the measurement may be output.

The quality determined in step 415, the first physical parameters determined in step 405, and/or the second physical parameters determined in step 410 may be utilized in a step 455 for determining an item of information regarding the determination quality. This makes it possible to output an item of information with regard to the determined quality during the measurement, in particular during time period 225. The item of information may be output in step 455, possibly together with a suggestion for improving the determination quality.

1-10. (canceled)

11. A device for determining a concentration of a predefined gas in a medium, the device comprising:
a first sensor for determining a gas concentration in a flow of the medium;
a second sensor for determining a physical parameter of the medium in the flow of the medium; and
a processing device configured to determine a quality of the determination by the first sensor on the basis of a signal from the second sensor.

12. The device as recited in claim 11, wherein the processing device is configured to control the determination of the gas concentration by the first sensor as a function of the determined quality.

13. The device as recited in claim 11, wherein a plurality of second sensors is provided, the sensors being configured to determine different physical parameters of the medium in the flow, and the processing device being configured to determine the quality of the determination on the basis of the signals from the second sensors.

14. The device as recited in claim 11, wherein a third sensor for determining a physical parameter in the environment of the device is provided, and the processing device is configured to determine the quality of the determination on the basis of the signal from the third sensor.

15. The device as recited in claim 11, wherein the processing device is configured to output an item of information regarding the determined quality.

16. The device as recited in claim 11, wherein the processing device is configured to supply an item of information regarding a more optimal handling of the device on the basis of the determined quality and a signal from the second and/or a third sensor, which is configured to determine a physical parameter in the environment of the device.

17. A method for determining the concentration of a predefined gas in a medium, the method comprising:
determining a gas concentration in a flow of the medium with the aid of a first sensor;
determining a physical parameter of the medium in the flow of the medium using a second sensor; and
determining a quality of the determination by the first sensor on the basis of a signal from the second sensor.

18. The method as recited in claim 17, wherein the determination of the gas concentration is found to be successful if a physical parameter of the medium lies within a predefined range during a measuring operation with the aid of the first sensor.

19. The method as recited in claim 17, wherein the determination of the gas concentration is found to be successful if a derivation of a physical parameter of the medium as a function of time lies within a predefined range during a measuring operation with the aid of the first sensor.

20. The method as recited in claim 17, wherein the determination of the gas concentration is found to be successful if a higher derivation of a physical parameter of the medium as a function of time lies within a predefined range during a measuring operation with the aid of the first sensor.
